(19) United States
(12) Patent Application Publication
(10) Pub. No.: US 2005/0208495 A1
(43) Pub. Date: Sep. 22, 2005

(54) Real-time detection of nucleic acid reactions

(76) Inventors: Richard A. Joseph, Stoughton, MA (US); James J. DiMeo, Needham, MA (US)

Correspondence Address: GIFFORD, KRASS, GROH, SPRINKLE & CITKOWSKI, P.C, PO BOX 7021, TROY, MI, 48007-7021, US

(21) Appl. No.: 10/528,288

(22) PCT Filed: Sep. 17, 2003
(86) PCT No.: PCT/US03/29418

§ 371 Date: Apr. 13, 2005

Related U.S. Application Data
(60) Provisional application No. 60/411,266, filed on Sep. 17, 2002.

(51) Int. Cl. C12Q 1/68

(52) U.S. Cl. 435/6

(57) ABSTRACT

A process is provided for using oligonucleotide to which a detectable moiety is attached post-synthesis. A metal-containing fluorescent compound affords real-time detection of nucleic acid elongation, amplification, or hybridization. The process is especially advantageous since a detectable moiety is readily attached to an existing oligonucleotide at an internal nucleotide, rather than being limited to attachment at a 3′ or 5′ terminus.

### RELATED APPLICATION

This application claims priority of U.S. Provisional Patent Application Ser. No. 60/411,266 filed Sep. 17, 2002, which is incorporated herein by reference.

### FIELD OF THE INVENTION

This invention relates to methods of detection of nucleic acids. In particular, the invention relates to methods of real-time fluorescence-based detection of changes in quantity, length and strandedness of a nucleic acid polymer.

### BACKGROUND OF THE INVENTION

Rapid detection and quantitation of nucleic acids is becoming increasingly important in basic research as well as in applied sciences. For example, many hospitals use polymerase chain reaction (PCR) to determine the identity of a pathogenic organism infecting a patient. Nucleic acid amplification techniques are also commonly used to assay environmental air and water samples suspected of contamination. Further, PCR is a key aspect of many forensic investigations. In each of these examples, it is important to obtain results as quickly and accurately as possible.

Various techniques have been developed to amplify nucleic acids including polymerase chain reaction, isothermal amplification, strand displacement amplification and ligase chain reaction. Detection and quantitation of amplified nucleic acids are currently limited by techniques that are time consuming and lacking in sensitivity. For instance, an amplification reaction may be subjected to gel electrophoresis followed by staining to visualize an approximate size and quantity of nucleic acid product. Gel detection often requires hours of processing before results are obtained.

Prior art methods of attaching a detectable moiety to an oligonucleotide include enzymatic incorporation of labeled nucleotides into a nucleic acid sequence, resulting in an oligonucleotide labeled at a terminus or in the internal portion of the molecule. Enzymatic incorporation techniques are inconvenient for labeling of internal nucleotides since the labeling must be performed during oligonucleotide synthesis. This precludes convenient storage of oligonucleotide stocks and on-demand labeling and use. End labeling techniques are also commonly used to incorporate a nucleotide attached to a detectable moiety and for direct bonding of a detectable label. However, many of these methods have the drawback that nucleotides must be derivatized in order to covalently bond the detectable label. Nucleotide derivatization and bonding of the label can interfere with oligonucleotide properties including ability to hybridize with specificity equal to an unlabeled oligonucleotide. Alternative methods allow bonding of a detectable moiety to an oligonucleotide at an internal nucleotide, but generally the choice of the nucleotide to which the linker is attached is limited. Further, steric hindrance is a common problem where bulky labels are attached, causing hybridization anomalies.

Thus, there exists a need for a method of detecting a nucleic acid of interest quickly and easily. Post-synthetic attachment of a detectable moiety to a nucleic acid would facilitate rapid detection.

### SUMMARY OF THE INVENTION

A process for detecting an oligonucleotide elongation involves the combination of a detectable moiety with an oligonucleotide through a non-covalent association. The resulting labeled oligonucleotide is added to an oligonucleotide elongation mixture in an elongation reaction thereafter initiated. Assaying for the labeled oligonucleotide for incorporation as part of the oligonucleotide elongation process affords the desired information. A fluorescent compound is considered a preferred detectable moiety.

Measurement of a fluorescence parameter in the oligonucleotide elongation reaction mixture at a first time point yields a test measurement. The comparison of the test measurement with a reference measurement affords oligonucleotide elongation detection.

A process for detecting oligonucleotide elongation includes providing oligonucleotide elongation reaction mixture containing an oligonucleotide labeled with a metal-containing fluorescent compound. Measurement of a fluorescence parameter associated with the metal-containing fluorescent compound in the reaction mixture at a first time point yields a test measurement. Comparison of the test measurement with a reference measurement affords oligonucleotide elongation detection. A platinum-containing fluorescent compound is particularly well suited to serve as the metal-Containing fluorescent compound.

A process for detecting formation of oligonucleotide hybrid includes providing a hybridization reaction mixture containing an oligonucleotide labeled with a metal-containing fluorescent compound. Measuring a fluorescence parameter associated with the metal-containing fluorescent compound at a first time point yields a test measurement associated with the reaction mixture. Comparison of the test measurement with a reference measurement affords oligonucleotide hybridization detection.

A commercial package includes a metal-containing fluorescent compound reaction mixture component along with instructions for use thereof to detect changes in an oligonucleotide indicative of elongation of hybridization. The use of a detectable moiety attached post-synthesis to an oligonucleotide for real-time detection of changes in nucleic acid elongation or hybridization is also provided.

### DETAILED DESCRIPTION OF THE PREFERRED EMBODIMENTS

The present invention provides methods for detecting and quantifying nucleic acids that overcome the limitations of prior technologies. In particular, the present invention provides methods of using a labeled nucleic acid oligonucleotide for real-time detection of changes in the oligonucleotide indicative of elongation and/or hybridization. The invention further provides methods for quantification of a nucleic acid of interest.

A method of the present invention for detecting oligonucleotide changes and/or quantitating a nucleic acid target includes the step of providing an oligonucleotide. Characteristics of a provided oligonucleotide sequence such as length, sequence and base composition depend on the type of reaction to be performed and the target nucleic acid to be detected. Typical reactions performed include an elongation reaction and a hybridization. Elongation reactions include, for example, a reverse transcription reaction and a polymerase chain reaction. It is appreciated that elongation reactions may include both a hybridization step and an elongation step and these may be detected separately according to an inventive method. A hybridization reaction may include formation of a DNA:DNA, DNA:RNA or RNA:RNA complex between a provided labeled oligonucleotide and a target nucleic acid. The target nucleic acid is any nucleic acid that a user desires to detect, for example, genomic DNA, mitochondrial DNA, total RNA, mRNA, tRNA and synthetic nucleic acids. Characteristics of an oligonucleotide suitable for these and related reactions are known in the art and are detailed in Sambrook et al., Molecular Cloning: A Laboratory Manual, Cold Spring Harbor Laboratory, 3^{Rd} Edition, 2001 and Dieffenbach, C. W. and Dveksler, G. S., PCR Primer: A Laboratory Manual, Cold Spring Harbor Laboratory, 1995.

An oligonucleotide to be used in an inventive method is labeled by attachment to a detectable moiety. A detectable moiety is a compound whose presence can be discovered upon application of an appropriate detection technique. For example, a detectable moiety is a fluorescent compound whose presence is discernable using techniques such as fluorimetry. Further examples of a detectable moiety include a biotin-containing compound, a compound containing an enzyme, such as horseradish peroxidase, or a radioactive compound. It is appreciated that in addition to excitation and direct detection of a fluorescent label according to the present invention, fluorescence resonance energy transfer (FRET) is operative herein with excitation of a first label moiety detected by fluorescence of a second label brought into proximity to the first label through elongation.

In order to overcome the limitations of the prior art, the present invention provides a process using an oligonucleotide in which a detectable moiety is attached post-synthesis. In a preferred embodiment, an oligonucleotide is attached to a detectable moiety that includes a fluorophore. Numerous fluorophores are known in the art including fluorescein, rhodamine, Cy-3, Cy-5, and others such as those listed in Handbook of Fluorescent Probes and Research Products, 8^{th} Edition (Molecular Probes, Eugene, Oreg.). It has been found that a metal-containing fluorescent compound used to label an oligonucleotide is particularly useful in a process for real-time detection of nucleic acid elongation, amplification, or hybridization. These fluorescent compounds are especially advantageous for use in an inventive process since a detectable moiety is readily attached to an existing oligonucleotide at an internal nucleotide, rather than being limited to attachment at the 5′ or 3′ terminus. In addition, a fluorescent compound is advantageous in not appreciably interfering with nucleic acid hybridization. Thus, a method according to the present invention allows a user to perform more rapidly the process of detecting a nucleic acid. The increased speed results from oligonucleotides of interest being stored until needed, quickly labeled, and used in a reaction, in which the product is detected by real-time changes in a fluorescent signal.

A particularly preferred label for an oligonucleotide used in an inventive method is a metal-containing fluorescent compound. Metals included in such compounds are the platinum group metals including platinum, palladium, rhodium, ruthenium, osmium, and iridium. For example, ULYSIS labels, such as ULYSIS Alexa Fluor 546 (Molecular Probes), are platinum-containing fluorescent compounds that are suitable labels for an oligonucleotide to be used in a method of the present invention, as detailed in the examples below. Further examples of suitable labels include those available commercially as Cy-Dye ULS fluorescent nucleic acid labels (Amersham) and those described in U.S. Pat. No. 6,338,943. Details of attaching a fluorescently labeled metal-containing compound to an oligonucleotide are found in Example 1 and in literature available from Molecular Probes including the Handbook of Fluorescent Probes and Research Products, 8^{th} Edition (Molecular Probes, Eugene, Oreg.).

In a method according to the present invention, the oligonucleotide is optionally labeled through a bond that is other than a covalent bond where each of the two bond atoms donates at least one electron to the bond, hereafter referred to as a “dual contribution covalent bond.” Binding through a bond other than a dual contribution covalent bond includes, for instance, formation of an ionic bond, hydrogen bond, Van der Waals interaction, and an organometallic coordinate covalent bond, between a compound including a detectable moiety and the oligonucleotide. An example of non-covalent binding that occurs as a combination of the above includes biological recognition interactions such as antibody/antigen binding.

Following oligonucleotide labeling, unlabeled oligonucleotide is preferably separated away from an unreacted detectable moiety. Separation is by a method known in the art such as use of a filtration column containing Sephadex or an art recognized equivalent as in Example 1. Suitable purification columns include those commercially available as ProbeQuant GSO Micro Column (Amersham Pharmacia Biotech, Piscataway, N.J.) and Label It Spin columns (PanVera).

In order to proceed with detection using an inventive method, a reaction mixture, such as an elongation, amplification or hybridization mixture, is prepared according to procedures known in the art. For instance, an elongation or amplification reaction may be a polymerase chain reaction, ligase chain reaction and, generally, reactions containing a nucleic acid polymerase. A typical PCR reaction mixture includes a first primer, which is fluorescently labeled as described above, a second primer that is optionally labeled, a nucleotide mix, a nucleic acid to be amplified and an enzyme. PCR reaction mixtures are known in the art and general guidelines regarding composition are found in Dieffenbach, C. W. and Dveksler, G. S., PCR Primer: A Laboratory Manual, Cold Spring Harbor Laboratory, 1995. A specific example is detailed in Example 2. A reaction mixture may be a hybridization mixture which typically includes a hybridization buffer, a nucleic acid target and a fluorescently labeled oligonucleotide probe. General guidelines regarding hybridization reaction mixtures are found in Sambrook et al., Molecular Cloning: A Laboratory Manual, Cold Spring Harbor Laboratory, 3^{rd} Edition, 2001.

Once a reaction mixture is assembled, an optional initial measurement of an oligonucleotide labeled with a detectable moiety in the reaction mixture is made. The measurement technique used depends on the detectable moiety used. In a preferred embodiment, a detectable moiety is a fluorescent compound, and measurement of a fluorescence parameter is made. Fluorescence parameters include, for example, fluorescence polarization and fluorescence intensity.

Fluorescence polarization is a particularly preferred mode of detection of nucleic acid changes according to the present invention. Fluorescence polarization measurements are used to detect differences in rotation of fluorescent molecules. Since a larger fluorescent molecule rotates more slowly than a smaller fluorescent molecule, changes in fluorescence polarization in a reaction including a fluorescently labeled oligonucleotide are indicative of changes in size of the oligonucleotide or its binding to another molecule. For example, fluorescence polarization changes are indicative of oligonucleotide binding to a polypeptide, hybridization with another nucleic acid sequence and elongation of the oligonucleotide. Fluorescence polarization measurements are made by directing polarized exciting light into a sample and measuring the polarized light emitted from the excited fluorophore. The technique is independent of fluorescence intensity as long as the signal is above the detection threshold of the detection equipment used. Various instruments are available commercially for measurement of fluorescence polarization. For example, a Victor2 V device (PerkinElmer Life Sciences, Boston, Mass.) is used to measure fluorescence polarization in an inventive method as described Example 2. Further general characteristics of methods and tools for fluorescence measurement are known in the art and are described in J. R. Lakowicz, Principles of Fluorescence Spectroscopy, 1999, 2^{nd} Edition. In addition, information relating to fluorescence polarization and nucleic acid detection is found in U.S. Pat. No. 6,022,686.

Changes in fluorescence intensity measurements in a nucleic acid amplification mixture correlate with changes in nucleic acid concentration. Fluorescence intensity measurements are made with any standard fluorimeter such as the Victor2 V device commercially available from PerkinElmer Life Sciences, Boston.

It is appreciated that multiple assays can be run in a single tube using oligonucleotides labeled with fluorophores having different excitation and/or emission spectra.

In methods to detect changes in a fluorescently labeled oligonucleotide, comparisons are made between a test measurement of a fluorescence parameter in the oligonucleotide-containing reaction mixture at a first time point and a reference. A reference may be a second measurement of a fluorescence parameter in the oligonucleotide reaction mixture at a second time point. The second time point may be before initiation of the reaction, i.e. to measure a basal level of a fluorescence parameter and to normalize for any background fluorescence. For example, in a PCR reaction, a reference measurement may be taken when an oligonucleotide is hybridized to target nucleic acid but before addition or activation of a polymerase.

Where a reference measurement is made in the reaction mixture prior to the reaction taking place, a subsequent step is reaction initiation. The type of initiation step depends on the type of reaction and the appropriate reaction initiation will be recognized by one skilled in the art. For example, where the reaction is a polymerase chain reaction, the reaction may be initiated by addition or activation of an enzyme such as Taq polymerase. A hybridization reaction is typically initiated by heating to dissociate double-stranded nucleic acid followed by bringing the mixture to incubation temperature.

It is apparent that more than two fluorescence measurements may be made and compared. In a method to detect an oligonucleotide elongation, such as occurs in a polymerase chain reaction, a first reference measurement may be made in a reaction mixture containing labeled oligonucleotide before the reaction is heated and cooled to dissociate any double-stranded nucleic acids present and hybridize the oligonucleotide to a target. Subsequently, a second reference measurement may be made once the oligonucleotide hybridization has taken place. Then, the polymerase chain reaction is initiated, for instance by addition of a suitable polymerase and a desired number of test measurements are made.

Alternatively, the first and second time points are after initiation of the elongation reaction.

Following reaction initiation, a test measurement of the reaction mixture is made. The reference and test measurements are then compared, resulting in an indication of changes in quantity, length and strandedness of a nucleic acid polymer such that an elongation, amplification or hybridization reaction is detected.

In an alternative embodiment, a reference is a measurement of a fluorescence parameter in a second oligonucleotide reaction mixture. For example, fluorescence measurements in a reaction mixture containing an unknown amount or type of nucleic acid sequence to be amplified may be compared to measurements in a reference reaction mixture in order to normalize for background or for quantitation. In an exemplary method, assessment of quantity of nucleic acid target is made by comparison to standard reactions containing known amounts of nucleic acid target. Standard reactions are preferably run in parallel with reactions containing an unknown amount of target. A standard curve relating amount of nucleic acid target and fluorescent signal is then generated and the amount of target nucleic acid present in the unknown sample is determined by comparison to the standard curve. Alternatively, a standard curve may be generated by using an internal standard as a reference. An internal standard may be a known amount of a nucleic acid added to a reaction mixture containing an unknown amount of a nucleic acid. The test and reference measurements may be made in parallel in the same reaction mixture.

A commercial package or kit is provided for detecting changes in an oligonucleotide indicative of oligonucleotide extension or hybridization. The kit includes reaction mixture components selected from, for example, nucleotides, a reaction buffer, a polymerase, a column for purification of a labeled oligonucleotide, nucleic acid purification reagents, standards and instructions for use of the components to detect changes in an oligonucleotide indicative of elongation or hybridization. In a preferred embodiment, a kit includes reaction mixture components and instructions on using an oligonucleotide labeled with a metal-containing fluorescent compound to detect changes in an oligonucleotide by detection of a fluorescence parameter.

### EXAMPLES

### Example 1

Labeling of Primer

To 5 micrograms of a tubulin forward primer is added 1 microliter ULYSIS TM Alexa Fluor 546 reagent (Molecular Probes, Eugene, Oreg.), brought up in 100 microliters of 50% dimethyl formamide, and 5 millimolar Tris-HCl pH 7.6 to a final volume of 20 microliters. The solution is heated at 85° C. for 30 minutes, and the volume is adjusted to 77 microliters with Tris buffer. The labeled primer is purified on a ProbeQuant G50 Micro Column (Amersham Pharmacia Biotech, Piscataway, N.J.). Final concentration is 10 micromolar.

### Example 2

Polymerase Chain Reaction and Detection

The PCR mixture is prepared as follows:

To 25 microliters of PCR Supermix with Platinum Taq (Invitrogen Life Technologies, Carlsbad, Calif.) is added 1 microliter of labeled primer from Example 1, 1 microliter of a 10 micromolar solution of unlabeled tubulin reverse primer and 1 microliter of a 5 picogram/microliter tubulin DNA solution. The solution is thermalcycled on an MJ Research PTC-100 thermal controller (MJ Research, Watertown, Mass.) at 95° C. for 3 minutes, and then 40 cycles of 95° C. for 20 seconds and 55° C. for 20 seconds, and then held at 4° C.

Twenty microliters of the PCR products and a control mixture where no PCR cycling is done are removed and placed into a well of an MJ 386 plate. Fluorescence polarization is measured in a Victor2 V (PerkinElmer Life Sciences, Boston, Mass.).

The control, or reference, mixture fluorescence polarization is 258 mP and the 40 cycle mixture fluorescence is 301 mP, for a 43 mP increase. This change in fluorescence polarization demonstrates that fluorescence polarization is a measure of primer elongation.

Aspects of fluorescence measurements in detection of nucleic acids are described in U.S. Pat. No. 6,022,686.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to obtain the ends and advantages mentioned, as well as those inherent therein. The present methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A process of detecting an oligonucleotide elongation, the process comprising:
(a) providing an oligonucleotide;
(b) combining a detectable moiety and the oligonucleotide to form a labeled oligonucleotide, the labeled oligonucleotide characterized by an association independent of a dual contribution covalent bond between the detectable moiety and the oligonucleotide;
(c) adding the labeled oligonucleotide to an oligonucleotide elongation mixture;
(d) initiating an elongation reaction in the oligonucleotide elongation mixture; and
(e) assaying for the labeled oligonucleotide in the oligonucleotide elongation mixture to detect the oligonucleotide elongation.

2. (canceled)

3. The process of claim 1 wherein the detectable moiety comprises a fluorophore.

4. The process of claim 1 wherein the detectable moiety comprises a metal-containing fluorescent compound.

5. The process of claim 4 wherein the metal-containing fluorescent compound comprises platinum.

6. The process of claim 4 wherein the metal-containing fluorescent compound comprises a metal selected from the group consisting of: palladium, rhodium, ruthenium, osmium, and iridium.

7. The process of claim 1 wherein the elongation reaction is a polymerase chain reaction.

8. The process of claim 1 wherein the elongation reaction is a reverse transcription reaction.

9. The process of claim 1 wherein the elongation reaction is a primer extension reaction.

10. The process of claim 1 wherein the elongation reaction is a ligase chain reaction.

11. The process of claim 1 wherein the process further comprises purifying the labeled oligonucleotide.

12. The process of claim 1 wherein the step of assaying the labeled oligonucleotide comprises a measurement selected from the group consisting of: fluorescence polarization, fluorescence intensity, and fluorescence resonance energy transfer.

13-14. (canceled)

15. A process of detecting an oligonucleotide elongation, the process comprising the steps of:
(a) providing an oligonucleotide elongation reaction mixture comprising an oligonucleotide labeled with a fluorescent compound by an association independent of a dual contribution covalent bond;
(b) measuring a fluorescence parameter in the oligonucleotide elongation reaction mixture at a first time point to obtain a test measurement; and
(c) comparing the test measurement with a reference measurement to detect the oligonucleotide elongation.

16. The process of claim 15 wherein the reference is a second measurement of a fluorescence parameter in the oligonucleotide reaction mixture at a second time point.

17. The process of claim 16 wherein the second time point is before initiation of the elongation reaction.

18. The process of claim 16 wherein the first and second time points are after initiation of the elongation reaction.

19. The process of claim 15 wherein the reference is a measurement of a fluorescence parameter in a second oligonucleotide extension reaction mixture.

20. (canceled)

21. The process of claim 15 wherein the fluorescent compound is a metal-containing fluorescent compound.

22. The process of claim 21 wherein the metal-containing fluorescent compound comprises platinum.

23. The process of claim 21 wherein the metal-containing fluorescent compound comprises a metal selected from the group consisting of: palladium, rhodium, ruthenium, osmium, and iridium.

24. The process of claim 15 wherein the elongation reaction is a polymerase chain reaction.

25. The process of claim 15 wherein the elongation reaction is a reverse transcription reaction.

26. The process of claim 15 wherein the elongation reaction is a primer extension reaction.

27. The process of claim 15 wherein the elongation reaction is a ligase chain reaction.

28. The process of claim 15 wherein the fluorescence parameter is selected from the group consisting of: fluorescence polarization and fluorescence intensity and fluorescence resonance energy transfer.

29-38. (canceled)

39. A process of detecting formation of an oligonucleotide hybrid, the process comprising:
(a) providing a hybridization reaction mixture comprising an oligonucleotide labeled with a metal-containing fluorescent compound;
(b) measuring a fluorescence parameter associated with the metal-containing fluorescent compound in the hybridization reaction mixture at a first time point to obtain a test measurement; and
(c) comparing the test measurement with a reference measurement to detect the oligonucleotide hybridization.

40-54. (canceled)
